(19) United States
(12) Patent Application Publication
(10) Pub. No.: US 2025/0290654 A1
(43) Pub. Date: Sep. 18, 2025

(54) VERTICAL DUCT FOR BUILDING AIR CONDITIONING SYSTEM

(71) Applicant: ISHIKAWA CO., LTD., Niigata-shi (JP)

(72) Inventor: Sachio ISHIKAWA, Niigata-shi (JP)

(21) Appl. No.: 19/066,107

(22) Filed: Feb. 27, 2025

(30) Foreign Application Priority Data
Mar. 12, 2024 (JP) 2024-037792
Jan. 14, 2025 (JP) 2025-004467

(51) Int. Cl. F24F 8/22; A61L 9/20; F24F 13/02

(52) U.S. Cl. CPC F24F 8/22; F24F 13/0227; A61L 9/205

(57) ABSTRACT

A vertical duct used for circulating air through spaces of upper and lower floors in a building, which is capable of keeping the air clean while maintaining it at appropriate temperature and humidity levels in the entire building, is to be provided. Photocatalyst is sprayed onto an inner wall surface of a vertical duct, and baffle plates are placed at regular intervals on a long shaft thereof in its longitudinal direction. Each of the baffle plates is formed to have a circular shape, and an LED lighting for emitting light toward the photocatalyst is provided on the inner wall surface of the vertical duct. An axial flow fan may be a vertical-duct fan including an HEPA filter and a scirocco fan.

### TECHNICAL FIELD

The present invention relates to a vertical duct for a building air conditioning system that controls the air to be in a uniform condition throughout a building, which is effective for cleaning the air.

### BACKGROUND ART

As disclosed in Patent Literature 1, the inventor has developed a building air conditioning system and method for controlling the air to be in a uniform condition throughout a building. In the technique disclosed therein, a vertical duct is used to circulate the air through the spaces on the lower and upper floors.

In the building air conditioning system according to Patent Literature 1, a house is considered as a single box, in which new air is introduced and widely circulated through the first and second floors by means of an axial flow fan, and a flow of air is created by means of ventilation fans provided, respectively, in the rooms that have been closed. Forcing the flow of air thus created to circulate through the building causes the air inside the building to be stirred up, and thus made in a uniform condition.

### CITATION LIST

### Patent Literature

[Patent Literature 1] JP-A-2022-74502

### SUMMARY OF INVENTION

### Technical Problem

In buildings, it is required to keep the air clean, for example, for the purpose of prevention of infectious diseases.

Here, focusing on the fact that titanium dioxide (photocatalyst) breaks down viruses in the air and inactive them, application of photocatalysts in a building air conditioning system to maintain the air inside the building clean has been a technical problem to be solved.

The present invention has been made in view of the circumstances described above, and thus an object thereof is to provide a vertical duct for a building air conditioning system to be used to circulate the air throughout the spaces of the upper and lower floors of a building, which is capable of keeping the air clean while maintaining it at the appropriate temperature and humidity throughout the building.

### Solution to Problem

In order to solve the problem described above, the preset invention provides, as a first aspect, a vertical duct for a building air conditioning system,
the vertical duct being provided for circulating an air through spaces of a lower floor and an upper floor in a building,
a photocatalyst being sprayed onto an inner wall surface of the vertical duct, and
the vertical duct comprising a plurality of baffle plates at regular intervals on a long shaft thereof in a longitudinal direction.

Furthermore, the present invention provides, as a second aspect, a vertical duct for a building air conditioning system,
the vertical duct being to be used for the building air conditioning system to circulate an air through spaces of a lower floor and an upper floor in a building,
the building air conditioning system being configured to keep a temperature and a humidity appropriately in all spaces of the building, the all spaces including a living space on the lower floor, a common space on the lower floor, a common space on the upper floor, at least one partitioned space on the lower floor, and at least one partitioned space on the upper floor,
a first air conditioner being installed in the living space on the lower floor and a second air conditioner being installed on one position of the common space on the upper floor,
air-path fans being installed horizontally on a wall of the at least one partitioned space on the lower floor and a wall of the at least one partitioned space on the upper floor, respectively, so as to communicate the living space on the lower floor, the common space on the lower floor, and the common space on the upper floor with the at least one partitioned space on the lower floor and the at least one partitioned space on the upper floor such that a fluid can move therethrough,
an axial flow fan being provided on a vertical portion of the vertical duct,
a photocatalyst being sprayed onto an inner wall surface of the vertical duct,
the vertical duct comprising a plurality of baffle plates at regular intervals on a long shaft thereof in a longitudinal direction, and
the vertical duct being provided vertically between the living space on the lower floor and one position of the common space on the upper floor, so as to communicate the living space on the lower floor with the common space on the upper floor and the at least one partitioned space on the upper floor such that a fluid can move therethrough.

Still further, the present invention provides, as a third aspect, the vertical duct for a building air conditioning system according to the first or second aspect, wherein
an LED lighting for emitting a light toward the photocatalyst is provided on the inner wall surface of the vertical duct.

Still further, the present invention provides, as a fourth aspect, the vertical duct for a building air conditioning system according to the first or second aspect, wherein
each of the baffle plates is formed to have a circular shape.

Still further, the present invention provides, as a fifth aspect, the vertical duct for a building air conditioning system according to the second aspect, wherein
the axial flow fan is a vertical-duct fan including an HEPA filter and a scirocco fan.

### Advantageous Effects of Invention

According to the vertical duct of the present invention, providing, in a route for circulating air appropriately in a building, an LED lighting and photocatalyst that has been sprayed onto the route, and making the air come into contact with the photocatalyst efficiently enables the air to be kept clean throughout the building.

### BRIEF DESCRIPTION OF DRAWINGS

FIG. 1 is a schematic longitudinal cross-section view of a building, in which a preferred embodiment of an overall structure of a building air conditioning system provided with a vertical duct for the building air conditioning system according to the present invention is illustrated.

FIG. 2 is a schematic longitudinal cross-section view of a building, in which a further preferred embodiment of an overall structure of a building air conditioning system provided with a vertical duct for the building air conditioning system according to the present invention is illustrated.

FIG. 3 is a schematic diagram illustrating an example of an air intake/exhaust port installed on a ceiling of a living space on the first floor illustrated in FIG. 1.

FIG. 4 is a schematic diagram illustrating an example of an air intake/exhaust port installed on a wall surface of a common space on the second floor illustrated in FIG. 1.

FIG. 5 is a schematic diagram illustrating an example of an axial flow fan positioned in a storage space of a common space on the second floor illustrated in FIG. 1.

FIG. 6 is a schematic diagram illustrating an example of an air intake/exhaust port of an air-path fan installed on a wall surface of a WC (toilet) on the first floor illustrated in FIG. 1.

FIG. 7 is a cross-section view of a longitudinal cross-section (illustrated in upper area) and a lateral cross-section (illustrated in lower area) of a preferred embodiment of a vertical duct according to the present invention.

FIG. 8 is a perspective view of a preferred embodiment of a vertical duct.

FIG. 9 is a longitudinal cross-section view of a preferred embodiment of an air-intake port.

FIG. 10 is a lateral cross-section view of a preferred embodiment of an air-intake port.

FIG. 11A is a top view of a vertical-duct fan as a preferred embodiment of an axial flow fan.

FIG. 11B is a front view of the vertical-duct fan illustrated in FIG. 11A.

FIG. 11C is a side view of the vertical-duct fan illustrated in FIG. 11B.

FIG. 12A is a top view of a partial cross-section illustrating an internal structure of the vertical-duct fan illustrated in FIG. 11.

FIG. 12B is a front view of a partial cross-section of that illustrated in FIG. 12A.

FIG. 12C is a side view of a partial cross-section of that illustrated in FIG. 12B.

FIG. 13 is a block diagram illustrating a preferred embodiment of sensors and a control unit of a building air conditioning system provided with a vertical duct according to the present invention.

### DESCRIPTION OF EMBODIMENTS

Hereinafter, a preferred embodiment of a vertical duct according to the present invention, which is to be provided in a building air conditioning system, will be described with reference to the drawings.

### <Design Concept of Air Conditioning System>

In the following, the design concept of an air conditioning system according to the present embodiment will be described.

Firstly, for example, the air conditioning system according to the present embodiment appropriately adjusts the temperature of air in a single space including a living room, a dining room, and a kitchen (such as a single space will be referred to as “LDK” herein), which serves as the center of life in a building. By circulating the air thus adjusted throughout the building, the variation in the temperature of air reduces in a corner area or near a wall of each room, so that the air can be controlled in a uniform condition throughout the building. In this case, the LDK functions as a room for heating or cooling.

Using only a conventionally used duct, the air cannot be circulated throughout the building as the volume of air flow is insufficient. According to the building air conditioning system of the present embodiment, not only an axial flow fan is provided in the vertical duct, but also an air-path fan is installed in each room, whereby the air can be circulated throughout the building.

Even if a heating operation is carried out locally in a typical Japanese building with thin exterior walls, the entire building cannot be heated to be a uniform condition as the temperature drops largely near the walls, windows, and peripheral portions of the building. According to the building air conditioning system of the present embodiment, circulating the air throughout all the spaces in the building including a narrow space such as a toilet enables the entire building to be uniformly heated.

Furthermore, according to the building air conditioning system of the present embodiment, considering humidity related to temperature, for example, the air with its humidity being adjusted by operating a humidifier in winter (dry season) while operating a dehumidifier in summer (humid season) is circulated throughout the building. This enables the humidity to be controlled throughout the building in a uniform condition at a desired humidity level, which is, for example, 40% to 60%. Even a system for houses with high heat insulation performance, which are common in the Western countries, does not have a function of adjusting the humidity uniformly throughout the building.

Controlling the temperature in a uniform condition throughout the building can prevent the temperature near the interior walls and glass portions from easily lowering. This can prevent the condensation from forming, and thus prevent the mold from growing. Thus, achieving the temperature and humidity which are gentle to the human bodies leads to creation of a healthy environment.

### <Basic Structure of Overall Air Conditioning System>

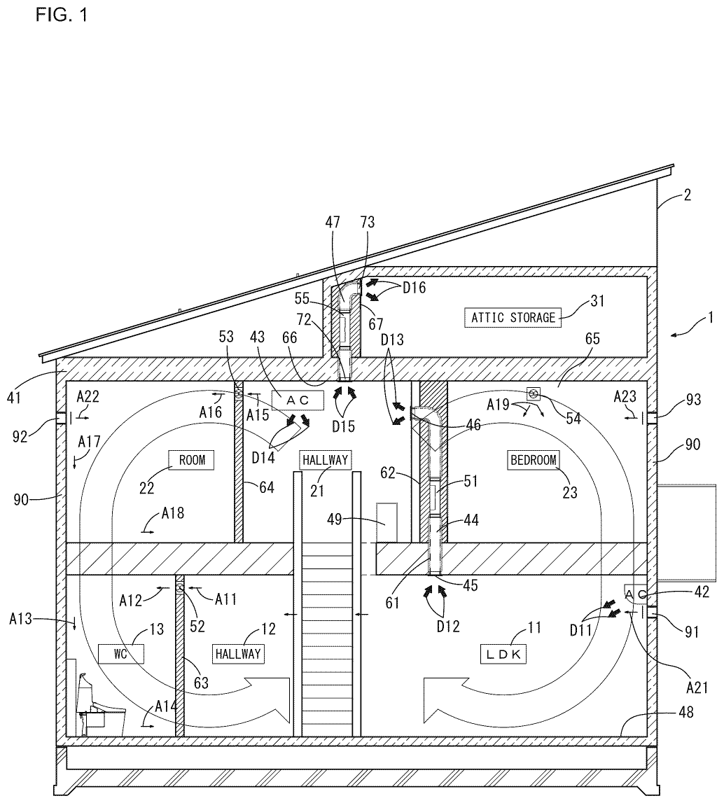

FIG. 1 is a schematic longitudinal cross-section view of a building, in which a preferred embodiment of an overall structure of a building air conditioning system provided with a vertical duct according to the present invention is illustrated.

An air conditioning system 1 according to the present embodiment is configured to keep the temperature and humidity appropriately in all spaces of a building. Here, all spaces of a building include, a living space on the lower floor, a common space on the lower floor, a common space on the upper floor, other partitioned spaces on the lower floor, and other partitioned spaces on the upper floor. According to the air conditioning system 1, the lower floor and the upper floor are connected with each other via a duct in which an air blower is appropriately arranged. The number of ducts to be provided may be minimum. According to the air conditioning system 1, by sending a large volume of air to every room and space and circulating the air throughout the building, the temperature and humidity of every room and space in the entire building can be controlled at appropriate levels.

A building 2 according to the present embodiment may have two floors or more, and in FIG. 1, the lower floor corresponds to the first floor and the upper floor corresponds to the second floor. In FIG. 1, an LDK 11 on the first floor is an example of the living space on the lower floor as mentioned above. In FIG. 1, a hallway 12 on the first floor is an example of the common space on the lower floor. In FIG. 1, a hallway 21 on the second floor is an example of the common space on the upper floor. In FIG. 1, a toilet (WC) 13 on the first floor is an example of the other partitioned spaces on the lower floor. In FIG. 1, a room 22 and a bedroom 23 on the second floor are examples of the other partitioned spaces on the upper floor. In the following, an example illustrated in FIG. 1 will be described.

The building 2 preferably has high air-tightness and thermal insulation. In FIG. 1, an insulation layer 41 is formed so as to surround the entire building. For example, the insulation layer 41 can be formed as the single insulation layer 41 by surrounding the entire building 2 with a thermal insulation material having high air tightness and thermal insulation. Preferably, the air tightness of the building 2 is C value=1.0 cm²/m² or less, and UA value=0.6 W/m² K or less.

In the LDK 11 on the first floor, an air conditioner 42 serving as a first air conditioner is installed. In the hallway 21 on the second floor, an air conditioner 43 serving as a second air conditioner is installed. Each of the first air conditioner and the second air conditioner may be any machine as long as it has at least a heating function and a cooling function. Alternatively, each of the first air conditioner and the second air conditioner may be formed with separate machines, one of which has only a heating function and the other of which has only a cooling function. The air conditioner 42 and the air conditioner 43 may be installed at any position, however, in particular, the air conditioner 43 to be installed in the hallway 21 on the second floor is preferably placed on a position where an air flow and ease of maintenance is considered. In which space the air conditioners to be installed and how many air conditioners to be installed in the spaces are not particularly limited, however, it is preferable to install one air conditioner in any space on the first floor and one air conditioner in any space on the second floor.

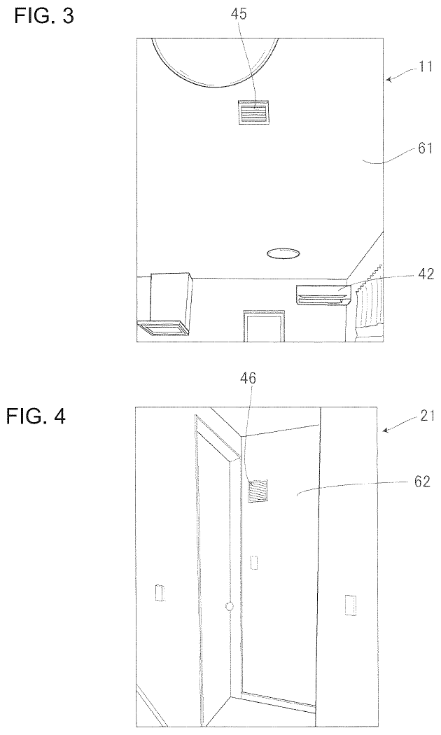

A vertical duct 44 is provided between the LDK 11 on the first floor and the hallway 21 on the second floor. Providing the vertical duct 44 allows the LDK 11 on the first floor to be communicated with the hallway 21 on the second floor, so that a fluid can move therethrough. An air intake/exhaust port 45 of the vertical duct 44, which is positioned toward the LDK 11 on the first floor, is provided on a ceiling 61 of the LDK 11 on the first floor. FIG. 3 is a schematic diagram illustrating an example of the air intake/exhaust port 45 installed on the ceiling 61 of the LDK 11 on the first floor. An air intake/exhaust port 46 of the vertical duct 44, which is positioned toward the hallway 21 on the second floor, is provided on a wall surface 62 of the hallway 21 on the second floor. FIG. 4 is a schematic diagram illustrating an example of the air intake/exhaust port 46 installed on the wall surface 62 of the hallway 21 on the second floor. Which space on the lower floor and which space on the upper floor are to be communicated with each other in a fluidic manner is not particularly limited, however, it is preferable to provide the vertical duct 44 so as to connect one of the spaces on the first floor where the air conditioner 42 is installed with one of the spaces on the second floor where the air conditioner 43 is installed.

The vertical duct 44 includes an axial flow fan 51 which is provided on its vertical portion. As the axial flow fan 51, for example, the Counter Arrow Fan (trademark registered in Japan) may be used.

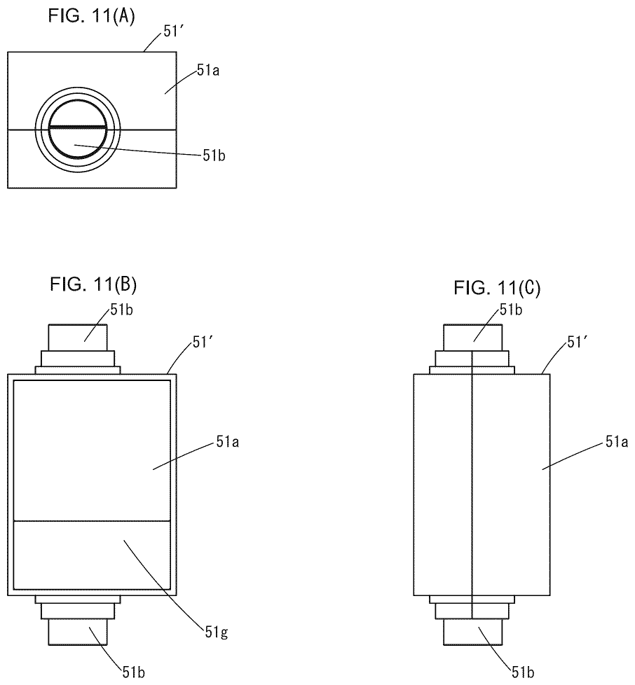

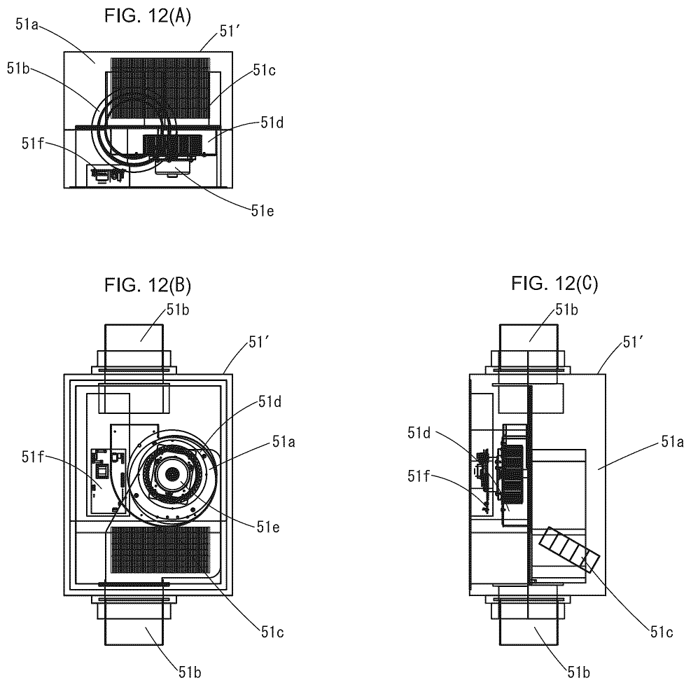

Alternatively and preferably, the axial flow fan 51 formed as a vertical-duct fan 51′ as illustrated in each of FIG. 11 and FIG. 12 may be provided in the vertical duct 44 (the same applies to a vertical duct 47 which will be described later).

The vertical-duct fan 51′ includes a casing 51a, and an HEPA (High Efficiency Particulate Air) filter 51c and a scirocco fan 51d with a motor 51e are provided inside the casing 51a. In addition, a control board 51f, vertical-duct connection ports 51b provided at the top and bottom of the casing 51a, respectively, and an opening panel 51g for maintenance are illustrated in each of FIG. 11 and FIG. 12.

Due to its low air-blowing performance, a conventional air purifier cannot send the air from the first floor to the second floor or from the second floor to the first floor in a building. For sending the air toward the upper floor and the lower floor to make the temperature and humidity therein uniform, the vertical-duct fan 51′ is designed to have increased performance, so that it has both the functions of an air purifier and those of a blower.

Thus, providing the HEPA filter 51c in the vertical-duct fan 51′ enables further improvement in the removal rate of airborne viruses, PM 2.5, and the like.

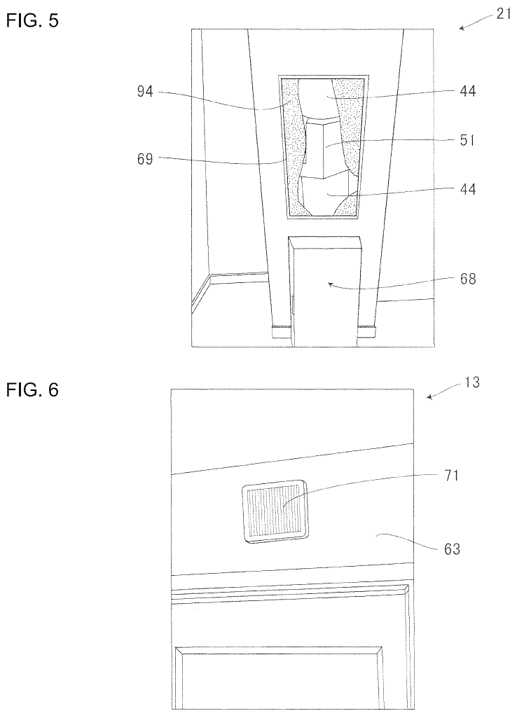

The axial flow fan 51 described above is mounted on a position where the LDK 11 on the first floor and the hallway 21 on the second floor are on a straight line. To which position of the vertical portion of the vertical duct 44 the axial flow fan 51 is to be mounted is not particularly limited, however, considering the ease of installation and maintenance, for example, the axial flow fan 51 may be mounted such that it is positioned in a storage space of the hallway 21 on the second floor. FIG. 5 is a schematic diagram illustrating the axial flow fan 51 positioned in the storage space of the hallway 21 on the second floor. FIG. 5 illustrates that a cover 68 for covering an opening 69 has been removed. Installation and maintenance for the axial flow fan 51 can be performed through the opening 69. The vertical duct 44 and the axial flow fan 51 are covered with a heat insulating material 94.

Air-path fans are mounted, for example, in each of the rooms, the washroom, and the toilet, respectively, to circulate the air throughout the building. In the present embodiment illustrated in FIG. 1, air-path fans 52, 53, 54 are installed horizontally on a wall 63 of the WC (toilet) 13 on the first floor, a wall 64 of the room 22 on the second floor, and a wall 65 of the bedroom 23 on the second floor, respectively. Providing the air-path fans 52, 53, 54 allows the LDK 11 on the first floor, the hallway 12 on the first floor, and the hallway 21 on the second floor to be communicated with the WC (toilet) 13 on the first floor, the hallway 22 on the second floor, and the bedroom 23 on the second floor, so that a fluid can move therethrough. FIG. 6 is a schematic diagram illustrating an example of an air intake/exhaust port 71 of the air-path fan 52 installed on the wall 63 of the WC (toilet) 13 on the first floor. As mounting ducts (not illustrated) for the air-path fans 52, 53, 54, for example, steel pipes may be used in view of fire prevention.

The air-path fan 52 is typically designed to send the air from the LDK 11 on the first floor and the hallway 12 on the first floor to the WC (toilet) 13 on the first floor. The air-path fan 53 is typically designed to send the air from the hallway 21 on the second floor to the room 22 on the second floor. The air-path fan 54 is typically designed to send the air from the hallway 21 on the second floor to the bedroom 23 on the second floor.

On an exterior wall 90 of the building 2, air intake ports 91, 92, 93 for introducing the outside air into the building 2 are provided. In FIG. 1, the air intake ports 91, 92, 93 are provided in the LDK 11 on the first floor, the room 22 on the second floor, and the bedroom 23 on the second floor, respectively, however, in which spaces the air intake ports are to be provided or and the number of the air intake ports to be provided are not particularly limited.

### <Structure of Vertical Duct>

In the following, the vertical duct according to the present embodiment will be described.

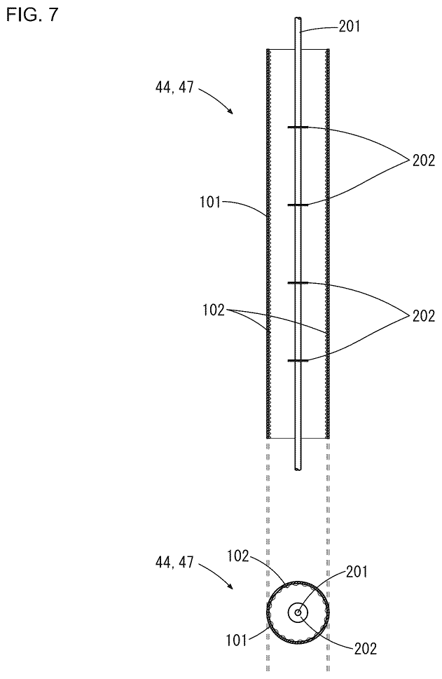

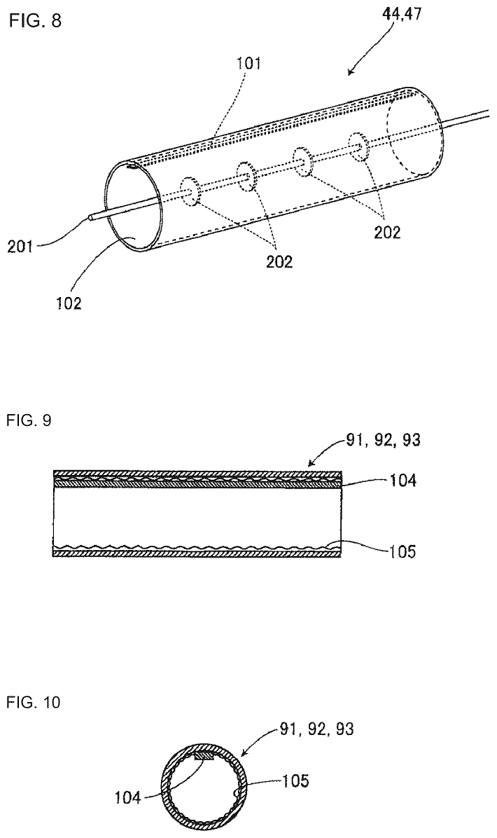

As illustrated in FIG. 7 and FIG. 8, the vertical duct 44 includes an LED lighting 101 on its inner wall surface, and photocatalyst 102 is sprayed onto the inner wall surface. The light emitted from the LED lighting 101 causes the photocatalyst 102 to function as a catalyst. The LED lighting 101 is not limited to a particular type, and for example, an LED in the form of a tape may be used and attached to the wall surface. The brightness of the LED lighting 101 is not limited to a particular level, however, preferably, it may be set to around, for example, 320 lm (lumens). The photocatalyst 102 is not limited to a particular type, and for example, titanium oxide may be used therefor.

In the vertical duct 44, baffle plates 202 are arranged horizontally at regular intervals on a long shaft 201 in its longitudinal direction. The long shaft 201 is, preferably, the center shaft of the vertical duct 44 in its longitudinal direction. The baffle plates 202 are not limited to particular shapes and thickness, and for example, they may be formed into the circular shapes as illustrated in FIG. 7 and FIG. 8, or the polygonal shapes such as triangles and squares. The baffle plates 202 may be fixed on, for example, a long rod which is placed on the long shaft 201. With this structure, the air resistance increases in the vertical duct 44, and by adjusting the airflow, the efficiency when the air comes into contact with the photocatalyst 102 can be improved.

### <Structure of Air Intake Port>

As illustrated in each of FIG. 9 and FIG. 10, an LED lighting 104 is provided on an inner wall surface of each of the air intake ports 91, 92, 93, and photocatalyst 105 is sprayed onto the inner wall surface. The light emitted from the LED lighting 104 causes the photocatalyst 105 to function as a catalyst. The LED lighting 104 is not limited to a particular type, and for example, an LED in the form of a tape may be used and attached to the inner wall surface. The brightness of the LED lighting 104 is not limited to a particular level, however, preferably, it may be set to around, for example, 320 lm (lumens). The photocatalyst 105 is not limited to a particular type, and for example, titanium oxide may be used therefor.

### <Flow of Air in Building>

In the following, the flow of air in the building according to the present embodiment will be described.

The air conditioner 42 installed in the LDK 11 on the first floor adjusts the temperature of the air in the LDK 11 on the first floor (see arrows D11). Due to the action of the axial flow fan 51, the air as adjusted flows to the hallway 21 on the second floor through the vertical duct 44 (see arrows D12, D13). In the same manner, the air as adjusted passes through the hallway 12 on the first floor, and flows to the WC (toilet) 13 on the first floor through the air-path fan 52 (see arrows A11, A12). The air flown into the WC (toilet) 13 on the first floor spreads to every corner of the WC (toilet) 13 on the first floor (see arrows A13, A14). Furthermore, the outside air is introduced from the air intake port 91 to the LDK 11 on the first floor (see arrow A21).

The air flown from the LDK 11 on the first floor to the hallway 21 on the second floor and the air adjusted by the air conditioner 43 installed in the hallway 21 on the second floor (see arrows D14) flow from the hallway 21 on the second floor to the room 22 on the second floor through the air-path fan 53 (see arrows A15, A16), and flows from the hallway 21 on the second floor to the bedroom 23 on the second floor through the air-path fan 54 (see arrows 19). The air flown in the room 22 on the second floor and the air flown in the bedroom 23 on the second floor spread to every corner of the room 22 on the second floor and every corner of the bedroom 23 on the second floor, respectively (see arrows A17, A18). The outside air is introduced from the air intake ports 92, 93 to the room 22 on the second floor and the bedroom 23 on the second floor, respectively (see arrows A22, A23).

As described above, the flow of air is formed through the LDK 11 on the first floor, the hallway 12 on the first floor, the WC (toilet) 13 on the first floor, the hallway 21 on the second floor, the room 22 on the second floor, and the bedroom 23 on the second floor, which allows the air in the building 2 to be circulated throughout the building 2. Furthermore, providing the photocatalyst 102, 105 in the path of the flow of air and causing the photocatalyst 102, 105 to function as catalysts using the LED lightings 101, 104 enables the air in the entire building 2 to be kept clean.

### <Reverse Rotation of Axial Flow Fan>

The flow of air described above can be changed by switching the rotation direction of the axial flow fan 51 from forward to reverse, or from reverse to forward. The rotation direction between forward and reverse may be switched manually, for example, using a control switch (not illustrated), or automatically using, if provided, a control unit 81 which will described below.

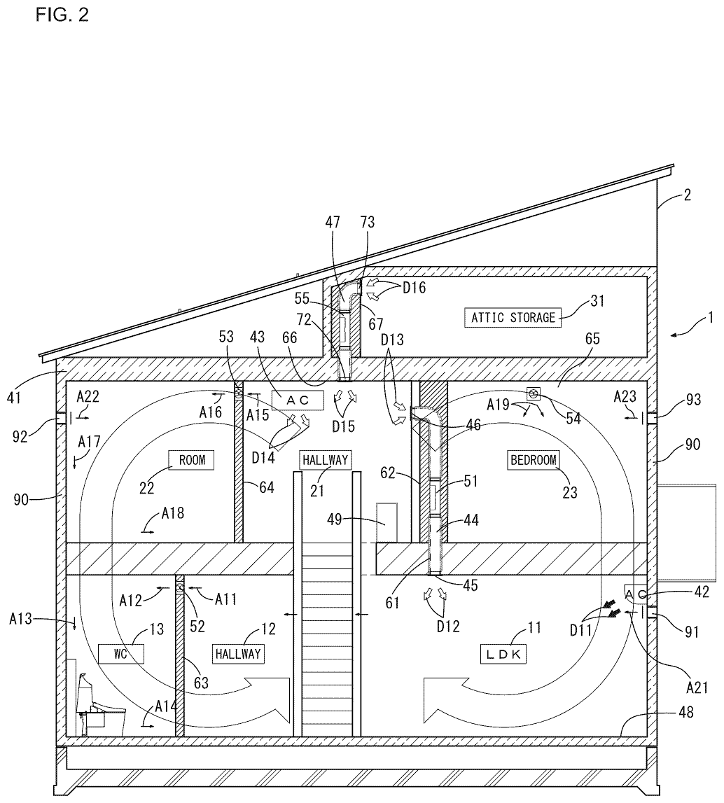

FIG. 2 is a schematic longitudinal cross-section view of a building, in which a further example of a preferred embodiment of the overall configuration of the air conditioning system 1 is illustrated. The basic structure of the air conditioning system 1 illustrated in FIG. 2 is the same as that illustrated in FIG. 1, however, the direction of air flowing through the vertical duct 44 by the axial flow fan 51 illustrated in FIG. 2 is different from that illustrated in FIG. 1. In FIG. 2, rotating the axial flow fan 51 in the direction which is opposite to the rotation direction according to the embodiment illustrated in FIG. 1 causes the air in the hallway 21 on the second floor to flow into the LDK 11 on the first floor (see arrows D23, D22).

The direction of air flowing through the vertical duct 44 is not limited to a particular direction, however, for example, in the case of operating the air conditioners 42, 43 in cooling modes, as illustrated in FIG. 1, the axial flow fan 51 may be set to cause the air to flow from the LDK 11 on the first floor to the hallway 21 on the second floor, while in the case of operating the air conditioners 42, 43 in heating modes, as illustrated in FIG. 2, the axial flow fan 51 may be set to cause the air to flow from the hallway 21 on the second floor to the LDK 11 on the first floor.

In the further embodiment illustrated in FIG. 2, the direction of air flowing through each of the air-path fans 52, 53, 54 is not different from that illustrated in FIG. 1, however, in the same manner as the axial flow fan 51, the rotation direction of each of the air-path fans 52, 53, 54 may also be switched between forward and reverse manually or automatically.

It is preferable to operate the air conditioners 42, 43 constantly all year round. This enables the temperature and humidity to be constantly kept appropriately throughout the year in all the spaces including the LDK 11 on the first floor, the hallway 12 on the first floor, the WC (toilet) 13 on the first floor, the hallway 21 on the second floor, the room 22 on the second floor, and the bedroom 23 on the second floor.

### <Attic Storage>

If the building 2 includes, for example, an attic storage 31 as a partitioned space on a floor further above the hallway 21 on the second floor, a vertical duct 47 with its vertical portion being provided with an axial flow fan 55 may be installed between the hallway 21 on the second floor and the attic storage 31. In FIG. 1, air intake/exhaust ports 72, 73 of the vertical duct 47 are positioned on a ceiling 66 of the hallway 21 on the second floor and a wall 67 of the attic storage 31, respectively. Preferably, the vertical duct 47 may be provided so as to connect any of the spaces on the second floor where the air conditioner 43 is installed with the partitioned space on the floor above the space.

As the axial flow fan 55, for example, the Counter Arrow Fan may be used.

Alternatively and preferably, the axial flow fan 55 formed as the vertical-duct fan 51′ as illustrated in each of FIG. 11 and FIG. 12 may be used (not described in detail here).

The axial flow fan 55 (or the vertical-duct fan 51′, the same applies to below) is mounted on a position where the hallway 21 on the second floor and the attic storage 31 are on a straight line. To which position of the vertical portion of the vertical duct 44 the axial flow fan 55 is to be mounted is not particularly limited, however, considering the ease of installation and maintenance, for example, the axial flow fan 55 may be mounted such that it is positioned in a storage space of the attic storage 31.

The axial flow fan 55 typically causes the air in the hallway 21 on the second floor to flow into the attic storage 31 as illustrated in each of FIG. 1 and FIG. 2 (see arrows D15, D16). In the same manner as the axial flow fan 51, the rotation direction of the axial flow fan 55 may also be switched between forward and reverse manually or automatically.

The structure in the vertical duct 47 may be the same as that in the vertical duct 44. The vertical duct 47 includes the LED lighting 101 on its inner wall surface, and the photocatalyst 102 is sprayed onto the inner wall surface (FIG. 7 and FIG. 8). The light emitted from the LED lighting 101 causes the photocatalyst 102 to function as a catalyst. The LED lighting 101 is not limited to a particular type, and for example, an LED in the form of a tape may be used and attached to the inner wall surface. The brightness of the LED lighting 101 is not limited to a particular level, however, preferably, it may be set to around, for example, 320 lm (lumens). The photocatalyst 102 is not limited to a particular type, and for example, titanium oxide may be used therefor.

In the vertical duct 47, baffle the plates 202 are arranged at regular intervals on the long shaft 201 in its longitudinal direction. The long shaft 201 is, preferably, the center shaft of the vertical duct 47 in its longitudinal direction. The baffle plates 202 are not limited to particular shapes and thickness, and for example, they may be formed into the circular shapes, or the polygonal shapes such as triangles and squares. The baffle plates 202 may be fixed on, for example, a long and thin rod which is placed on the long shaft 201. With this structure, the air resistance increases in the vertical duct 47, and by adjusting the airflow, the efficiency when the air comes into contact with the photocatalyst 102 can be improved.

### <Underfloor Heating System>

An underfloor heating system 48 may be installed at least in the LED 11 on the first floor. At least one of the air conditioner 42 and the underfloor heating system 48 warms the air in the LED 11 on the first floor to allow the temperature of the air to be controlled. The underfloor heating system 48 may be any conventional type, for example, a warm water heating system or an electric heating system.

### <Adjustment of Humidity>

A humidifier and dehumidifier for adjusting the humidity may be installed in at least one space in the building. As a device acting as a humidifier and dehumidifier, for example, a humidifying air cleaner 49 may be used. In each of FIG. 1 and FIG. 2, the humidifying air cleaner 49 is placed on the floor of the hallway 21 of the second floor. The air conditioning system 1 described above circulates the air, whose humidity has been conditioned in the hallway 21 on the second floor, throughout the building 2 to allow the humidity to be controlled at a uniform and constant level throughout the building 2. In which space the humidifying air cleaner 49 is to be placed and how many humidifying air purifiers 49 to be installed are not particularly limited, however, for example, installing the humidifying air purifier 49 in a space where the air conditioner is installed enables increase in the efficiency.

### <Wind Speed Inside Building>

Setting the wind speed inside the building 2 in the range of 0.15 to 0.25 m/sec prevents the residents from feeling uncomfortable. Thus, it is preferable to circulate the air throughout the building 2 so that the wind speed inside the building 2 falls within the range of 0.15 to 0.25 m/sec. Circulating the air using the air conditioning system 1 according to the present embodiment enables the wind speed inside the building 2 to be controlled in the range of 0.15 to 0.25 m/sec.

### <Control Unit>

In the following, the control unit according to the present embodiment will be described.

For automatically controlling the temperature, humidity, and wind speed in the building 2, the control unit 81 may be provided. The control unit 81 is configured to control the axial flow fans 51, 55, the air-path fans 52, 53, 54, the air conditioners 42, 43, and the humidifying air cleaner 49, which are included in the air conditioning system 1 according to the present embodiment.

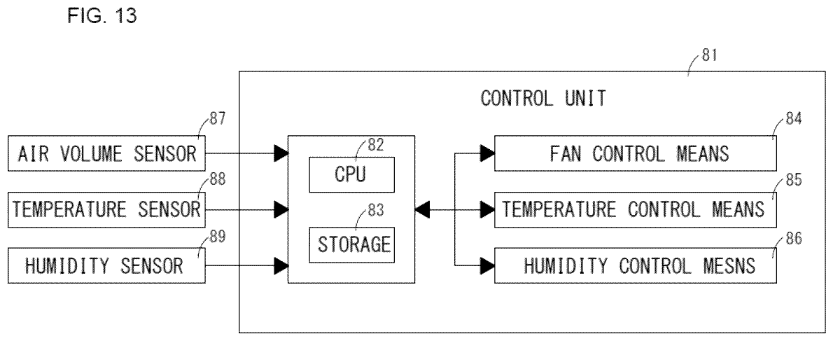

FIG. 13 is a block diagram illustrating a preferred embodiment of sensors and the control unit 81. For example, for controlling the axial flow fans 51, 55 and the air-path fans 52, 53, 54, the control unit 81 is configured such that a fan control means 84 controls the power on/off, rotation direction, wind speed, and the like of each of the axial flow fans 51, 55 and the air-path fans 52, 53, 54, depending on the condition of the air in the building 2. For controlling the air conditioners 42, 43, the control unit 81 is configured such that a temperature control means 85 controls the power on/off, switching of heating and cooling, wind speed, and the like of each of the air conditioners 42, 43, depending on the condition of the air in the building 2. For controlling the humidifying air cleaner 49, the control unit 81 is configured such that a humidity control means 86 controls the power on/off, switching of humidifying and dehumidifying, wind speed, and the like of the humidifying air cleaner 49, depending on the humidity in the building 2. In measurement of the wind speed, temperature, and humidity, for example, an air volume sensor 87, a temperature sensor 88, and a humidity sensor 89, may be used, respectively. Each of the sensors 87, 88, 89 may be installed in a predetermined space considering, for example, the necessity and effectiveness of measurement. The control unit 81 is configured with at least a CPU (Central Processing Unit) 82 and a storage 83, and implements the functions of each of the control means 84, 85, 86 mentioned above based on the input from each of the sensors 87, 88, 89. Alternatively, each of the control means 84, 85, 86 may be configured with a CPU and a storage.

The present invention has been described based on the embodiment, however, it is not limited to the examples illustrated in the drawings but includes any design change and variations which would be made by those ordinally skilled in the art within the scope not deviating from its technical concept. For example, the arrangement of the spaces and rooms in the building is not limited to the ones illustrated in FIG. 1 and FIG. 2. The number of vertical ducts, air intake ports, and air conditioners to be provided and the installation positions thereof can be set appropriately depending on the layout in the building to which the air conditioning system is to be applied. Furthermore, an air conditioning system is not limited to the one according to the present embodiment, and the vertical ducts according to the present invention are applicable to various building air conditioning systems.

### REFERENCE SIGNS LIST

1 air conditioning system
2 building
11 LDK on first floor (living space on lower floor)
12 hallway on first floor (common space on lower floor)
13 toilet (WC) (other partitioned spaces on lower floor)
21 hallway on second floor (common space on upper floor)
22 room on second floor (other partitioned spaces on upper floor)
23 bedroom on second floor (other partitioned spaces on upper floor)
31 attic storage (partitioned space on further upper floor)
41 insulation layer
42 air conditioner (first air conditioner)
43 air conditioner (second air conditioner)
44, 47 vertical duct
48 underfloor heating system
49 humidifying air cleaner
51, 55 axial flow fan
51′ vertical-duct fan
51a casing
51b vertical-duct connection port
51c HEPA filter
51d scirocco fan
51e motor
51f control board
51g opening panel for maintenance
52, 53, 54 air-path fan
81 control unit
84 fan control means
85 temperature control means
86 humidity control means
87 air volume sensor
88 temperature sensor
89 humidity sensor
90 exterior wall
91, 92, 93 air intake port
101, 104 LED lighting
102, 105 photocatalyst
201 long shaft (rod)
202 baffle plate

1. A vertical duct for a building air conditioning system,
the vertical duct being provided for circulating an air through spaces of a lower floor and an upper floor in a building,
a photocatalyst being sprayed onto an inner wall surface of the vertical duct, and
the vertical duct comprising a plurality of baffle plates at regular intervals on a long shaft thereof in a longitudinal direction.

2. A vertical duct for a building air conditioning system,
the vertical duct being to be used for the building air conditioning system to circulate an air through spaces of a lower floor and an upper floor in a building,
the building air conditioning system being configured to keep a temperature and a humidity appropriately in all spaces of the building, the all spaces including a living space on the lower floor, a common space on the lower floor, a common space on the upper floor, at least one partitioned space on the lower floor, and at least one partitioned space on the upper floor,
a first air conditioner being installed in the living space on the lower floor and a second air conditioner being installed on one position of the common space on the upper floor,
air-path fans being installed horizontally on a wall of the at least one partitioned space on the lower floor and a wall of the at least one partitioned space on the upper floor, respectively, so as to communicate the living space on the lower floor, the common space on the lower floor, and the common space on the upper floor with the at least one partitioned space on the lower floor and the at least one partitioned space on the upper floor such that a fluid can move therethrough,
an axial flow fan being provided on a vertical portion of the vertical duct,
a photocatalyst being sprayed onto an inner wall surface of the vertical duct,
the vertical duct comprising a plurality of baffle plates at regular intervals on a long shaft thereof in a longitudinal direction, and
the vertical duct being provided vertically between the living space on the lower floor and one position of the common space on the upper floor, so as to communicate the living space on the lower floor with the common space on the upper floor and the at least one partitioned space on the upper floor such that a fluid can move therethrough.

3. The vertical duct for a building air conditioning system according to claim 1, wherein
an LED lighting for emitting a light toward the photocatalyst is provided on the inner wall surface of the vertical duct.

4. The vertical duct for a building air conditioning system according to claim 1, wherein
each of the baffle plates is formed to have a circular shape.

5. The vertical duct for a building air conditioning system according to claim 2, wherein
the axial flow fan is a vertical-duct fan including an HEPA filter and a scirocco fan.

6. The vertical duct for a building air conditioning system according to claim 2, wherein
an LED lighting for emitting a light toward the photocatalyst is provided on the inner wall surface of the vertical duct.

7. The vertical duct for a building air conditioning system according to claim 2, wherein
each of the baffle plates is formed to have a circular shape.
